(19) United States
(12) Patent Application Publication
(10) Pub. No.: US 2023/0235020 A1
(43) Pub. Date: Jul. 27, 2023

(54) PHARMACEUTICAL COMPOUND FOR THE TREATMENT OF ATHEROSCLEROTIC CARDIOVASCULAR DISEASE

(71) Applicant: Ferring B.V., Hoofddorp (NL)

(72) Inventors: Dominik Schulte, Kiel (DE); Georg Wätzig, Kiel (DE); Matthias Laudes, Kiel (DE); Stefan Schreiber, Kiel (DE)

(21) Appl. No.: 18/009,469

(22) PCT Filed: Jun. 9, 2021
(86) PCT No.: PCT/EP2021/065407

§ 371 Date: Dec. 9, 2022

(30) Foreign Application Priority Data
Jun. 10, 2020 (EP) 20179285.0

(51) Int. Cl. C07K 14/715; A61P 9/10

(52) U.S. Cl. CPC C07K 14/7155; A61P 9/10; C07K 2319/30

(57) ABSTRACT

The invention provides a polypeptide dimer comprising two gp130-Fc fusion peptides for use in the treatment of ASCVD in human patients, preferably of high-risk ASCVD in human patients, more preferably of very-high-risk ASCVD in human patients.

### FIELD

The present invention relates to a polypeptide dimer comprising two gp130-Fc fusion peptides as its constituents for use in the treatment of atherosclerotic cardiovascular disease (ASCVD) in a human patient, as defined by the 2019 ESC/EAS Guidelines, particularly Table 4: Mach et al., Eur. Heart J. 41:111 (2020). The ASCVD comprises low density lipoprotein (LDL)-driven ASCVD, triglyceride-driven ASCVD, lipoprotein a-driven ASCVD, chronic inflammatory disease-driven ASCVD, or inflammatory ASCVD, and may be accompanied by one or more of familial hypercholesterolemia, chronic kidney disease, diabetes mellitus, blood pressure greater than 180/110 mm Hg, or human immunodeficiency virus infection.

Generally, the human patient can be a non-responder to treatment with or be intolerant to treatment with one or more of statin; ezetimibe; an inhibitor of proprotein convertase subtilisin/kexin type 9 (PCSK9), which preferably is a antibody such as alirocumab or evolocumab or a short interfering RNA, such as inclisiran; or lipid apheresis therapy.

### BACKGROUND

Inflammation is a strong driver of atherosclerotic cardiovascular disease (ASCVD) (Ross 1999, N. Engl. J. Med. 340:115). Patients with very-high-risk ASCVD (as defined by the 2019 ESC/EAS Guidelines, Table 4: Mach et al. 2020, Eur. Heart J. 41:111) and high inflammatory load despite state-of-the-art medical treatment have a large unmet need for effective therapies. Such treatments should prevent or reduce inappropriate inflammation while avoiding systemic immunosuppression (Ridker 2017, Circ. Res. 120:617), as this increases the risk of infections and does not reduce cardiovascular events (Ridker et al. 2019, N. Engl. J. Med. 380:752). Anti-cytokine therapy is a promising option for treating ASCVD that is progressive despite lifestyle modification and optimizing plasma lipid levels (Schuett & Schieffer 2012, Curr. Atheroscler. Rep. 14:187; Ait-Oufella et al. 2019, Arterioscler. Thromb. Vasc. Biol. 39:1510).

The recent CANTOS trial investigated the anti-interleukin-1β (IL-1β) antibody canakinumab in established human inflammatory ASCVD and demonstrated the challenge of significant benefits through lowering the rate of recurrent cardiovascular events at the expense of a higher incidence of fatal infections (Ridker et al. 2017, N. Engl. J. Med. 377:1119). Downstream of IL-113, interleukin-6 (IL-6) signaling is involved in atherogenesis (Scheller & Rose-John 2012, Lancet 380:338). IL-6 is a pleiotropic cytokine which is produced by haematopoietic and non-haematopoietic cells in response to infection and tissue damage. Patients with ASCVD show increased levels of circulating IL-6, which are correlated with clinical activity (Ridker et al. 2016, Circ. Res. 118:145). High IL-6 plasma levels are associated with a higher risk of future cardiovascular events (Kaptoge et al. 2014, Eur. Heart J. 35:578).

IL-6 exerts its multiple functions through two main signaling pathways, which both require signal transduction by a pre-formed dimer of the transmembrane co-receptor gp130 (Scheller et al. 2014, Semin. Immunol. 26:2). In classic signaling, IL-6 uses the membrane-bound IL-6 receptor (IL-6R), which is mainly expressed by hepatocytes and leukocytes. In the trans-signaling pathway, circulating soluble IL-6R (sIL-6R) produced by proteolytic cleavage or alternative splicing recruits IL-6 to form IL-6/sIL-6R complexes, which could activate the ubiquitously expressed gp130 on nearly any body cell (Garbers et al. 2018, Nat. Rev. Drug Discov. 17:395). Such ubiquitous trans-signaling is physiologically prevented by an excess of soluble gp130 isoforms (sgp130) acting as a buffer in the blood (Jostock et al. 2001, Eur. J. Biochem. 268:160). While classic IL-6 signaling has many physiological and anti-infectious functions, excessive trans-signaling is seen in many chronic inflammatory conditions. Specific trans-signaling inhibition instead of blocking IL-6 or its receptor has therefore been proposed to treat chronic inflammation without the negative effect of systemic immunosuppression (Rose-John et al. 2017, Nat. Rev. Rheumatol. 13:399; Garbers et al. 2018, Nat. Rev. Drug Discov. 17:395). As outlined above, inhibition of IL-1β by canakinumab led to a significantly lower rate of recurrent cardiovascular events and lowered IL-6 levels in humans. However, side effects due to the systemic immunosuppression by canakinumab led to an unfavourable risk/benefit ratio for the therapy of ASCVD (Ridker et al. 2017, N. Engl. J. Med. 377:1119; Palmer et al. 2019, Front. Cardiovasc. Med. 6:90)._ENREF_23 These results are in line with the increased rate of opportunistic and severe infections that is observed with the anti-IL-6R antibody tocilizumab (Rose-John et al. 2017, Nat. Rev. Rheumatol. 13:399). Another potential limitation of complete IL-6 inhibition is the potential increase in triglycerides and LDL cholesterol (Garbers et al. 2018, Nat. Rev. Drug Discov. 17:395).

EP 1 148 065 B1 and Jostock et al. 2001 (Eur. J. Biochem. 268:160) describe a fusion protein sgp130Fc that consists of two sgp130 domains fused to the crystallisable fragment of human immunoglobulin G1._ENREF_7 WO 2008/000516 A2 describes an optimized variant of sgp130Fc, which has received the international non-proprietary name olamkicept and is currently in clinical development by Ferring Pharmaceuticals (Saint-Prex, CH) and I-Mab Biopharma (Shanghai, CN).

Schuett et al. 2012 (Arterioscler. Thromb. Vasc. Biol. 32:281) showed that patients with coronary artery disease have lower plasma levels of endogenous sgp130 and described the reduction of atherosclerosis by sgp130Fc in a standard murine atherosclerosis model, which is genetically manipulated to lack the LDL receptor and fed a high-fat, high-cholesterol diet to maximise atherosclerotic disease. However, the translation of findings in such artificial murine genetic models to human diseases, which are influenced by a plethora of risk factors and behavioural variations, is frequently unsuccessful (Seok et al. 2013, PNAS 110:3507; Tsukamoto 2016, Drug Discov. Today 21:529) despite a correct choice of the disease model (Oppi et al. 2019, Front. Cardiovasc. Med. 6:46). For example, in the two most widely used genetic mouse models of atherosclerosis (Ldlr^{-/-} and Apoe^{-/-}), deletion of IL-6 can be atheroprotective (Madan et al. 2008, Atherosclerosis 197:504) and inhibition of IL-6R can reduce atherosclerotic lesions (Akita et al. 2017, Front. Cardiovasc. Med. 4:84). However, IL-6 elimination can also enhance rather than reduce atherosclerosis in exactly these models (Ramji & Davies 2015, Cytokine Growth Factor Rev. 26:673), underlining the complex physiological and pathophysiological functions of IL-6 signaling and the inherent uncertainties of murine models of complex, chronic disease.

### SUMMARY OF THE INVENTION

Patients with ASCVD frequently experience disease exacerbation and cardiovascular events despite maximum medical treatment. The problem is to provide a targeted anti-inflammatory therapy which diminishes local, LDL cholesterol-driven, self-perpetuating metabolic inflammation in atherosclerotic plaques without significant systemic immunosuppression.

The solution to this problem is provided by the features of the claims and especially by a polypeptide dimer comprising two gp130-Fc fusion peptides, exemplified by olamkicept, for use in the treatment of ASCVD in human patients, preferably of high-risk ASCVD in human patients, more preferably of very-high-risk ASCVD in human patients.

It has now been found that olamkicept can be administered to human patients diagnosed to have ASCVD, without any apparent side effects caused by the treatment. Surprisingly, the specific therapeutic inhibition of IL-6 trans-signaling by olamkicept in established atherosclerosis was found to reduce the atherosclerotic burden and local inflammatory activity in human patients with very-high-risk ASCVD with high efficacy, on an unexpectedly large scale and despite maximum medical treatment. The finding that olamkicept can provide a clinically significant regression of established atherosclerotic plaques and arterial wall inflammation in these patients despite optimized therapy and lifestyle is surprising, because the previously described effects of olamkicept in a murine model of atherosclerosis (Schuett et al. 2012, Arterioscler. Thromb. Vasc. Biol. 32:281) were obtained in a setting in which mice were genetically prone to severe atherosclerosis by artificial deletion of the LDL receptor, were fed a high-fat, high-cholesterol diet that massively induces atherosclerosis, and received olamkicept as the only medicament. In the human patients without the artificial deletion of the LDL receptor, however, olamkicept showed clinically meaningful effects as an additional therapy in an optimized therapeutic setting and was surprisingly able to beneficially influence key parameters of ASCVD obviously not appropriately targeted by the best available drugs against ASCVD, such as PCSK9 inhibitors or statins. Preferably, the key parameters are those defined by the 2019 ESC/EAS Guidelines (Mach et al. 2020, Eur. Heart J. 41:111).

The polypeptide dimer of the invention comprises two gp130-Fc monomers, each monomer having at least 90% sequence identity to SEQ ID NO: 1, preferably wherein the monomers comprise the gp130 D6 domain comprising the amino acids at positions 585-595 of SEQ ID NO:1, an Fc domain hinge region comprising the amino acids at positions 609-612 of SEQ ID NO:1, and, more preferred, the monomers do not comprise a linker between the gp130 part and the Fc part, but the gp130 part is directly linked to the Fc part, which is the case in the example of olamkicept. Further, the invention provides the polypeptide dimers, especially olamkicept, for use in methods of treatment of human patients diagnosed to have ASCVD, high-risk ASCVD or very-high-risk ASCVD.

Preferably, the human patients are non-responders to treatment with or intolerant to treatment with one or more of statin, ezetimibe, inhibitors of proprotein convertase subtilisin/kexin type 9 (PCSK9), or lipid apheresis therapy. Optionally, the human patients may suffer, e.g., from LDL cholesterol-driven ASCVD, triglyceride-driven ASCVD, lipoprotein a-driven ASCVD, chronic inflammatory disease-driven ASCVD, inflammatory ASCVD, familial hypercholesterolemia, chronic kidney disease, diabetes mellitus, blood pressure greater than 180/110 mm Hg, or human immunodeficiency virus infection.

### DETAILED DESCRIPTION OF THE INVENTION

The invention provides a polypeptide dimer, exemplified by olamkicept, for use in the treatment of ASCVD in human patients, preferably of high-risk ASCVD in human patients, more preferably of very-high-risk ASCVD in human patients. Herein, the polypeptide dimer comprises or consists of two gp130-Fc monomers, each monomer having at least 90% sequence identity to SEQ ID NO: 1, preferably wherein the monomers comprise the gp130 D6 domain comprising the amino acids at positions 585-595 of SEQ ID NO:1, an Fc domain hinge region comprising the amino acids at positions 609-612 of SEQ ID NO:1, and, more preferred, the monomers do not comprise a linker between the gp130 part and the Fc part.

The polypeptide dimer described herein inhibits excessive IL-6 trans-signalling by selectively targeting and neutralizing IL-6/sIL-6R complexes and is therefore considered to only inhibit IL-6 trans-signalling in the desired therapeutic concentrations, leaving classic signalling and its many physiological functions, as well as its acute inflammatory defence mechanisms, intact. Currently, the polypeptide dimers are found to have an efficacy similar to global IL-6 blockade, e.g., by the anti-IL-6R antibody tocilizumab or the anti-IL-6 antibody sirukumab, but with significantly fewer side effects, especially without general immunosuppression.

The polypeptide dimers described herein preferably comprise gp130-Fc monomers having the sequence corresponding to SEQ ID NO:1. In certain embodiments, polypeptide dimers described herein comprise polypeptides having at least 90%, 95%, 97%, 98%, 99% or 99.5% sequence identity to SEQ ID NO: 1. Preferably, the polypeptide dimers described herein comprise polypeptides having at least 90%, 95%, 97%, 98%, 99% or 99.5% sequence identity to amino acid positions 1-595 of SEQ ID NO: 1, corresponding to the gp130 sequence. Preferably, the Fc domain is an IgG1 or IgG4 Fc domain. Preferably, the polypeptide comprises the gp130 D6 domain (in particular the amino acid residues TFTTPKFAQGE: amino acid positions 585-595 of SEQ ID NO:1), the amino acid residues AEGA in the Fc domain hinge region (amino acid positions 609-612 of SEQ ID NO:1) and does not comprise a linker between the gp130 part and the Fc part. In a preferred embodiment, the disclosure provides a polypeptide dimer comprising two monomers having an amino acid sequence at least 90% sequence identify to SEQ ID NO: 1, wherein the amino acid sequence comprises the gp130 D6 domain, AEGA in the Fc domain hinge region, and there is no linker present between the gp130 part and the Fc part. In some embodiments, the invention provides compositions comprising a plurality of polypeptides described herein (e.g., a plurality of polypeptide monomers and/or polypeptide dimers described herein).

The polypeptide dimers of the invention are for use in parenteral administration, such as intravenous infusion or subcutaneous injection. Suitable formulations include those comprising a surfactant, particularly a nonionic surfactant such as a polysorbate surfactant (e.g., polysorbate 20). Formulations can also include buffering agents and sugars. An exemplary buffering agent is histidine. An exemplary sugar is sucrose. Thus, a suitable formulation could include polysorbate 20 (e.g., 0.01-1 mg/mL, 0.02-0.5 mg/mL, 0.05-0.2 mg/mL), histidine (e.g., 0.5 mM-250 mM, 1-100 mM, 5-50 mM, 10-20 mM) and sucrose (e.g., 10-1000 mM, 20-500 mM, 100-300 mM, 150-250 mM).

The polypeptide dimers of the invention are typically administered at doses of 60 mg-1 g, preferably 150 mg-600 mg. The dosing frequency is typically once every 1-4 weeks, preferably every 1-2 weeks.

The exemplification of the invention shows that olamkicept can be administered to patients with ASCVD without any significant side effects. Surprisingly, the specific therapeutic inhibition of IL-6 trans-signaling by olamkicept in established very-high-risk ASCVD (as defined by the 2019 ESC/EAS Guidelines, Table 4: Mach et al. 2020, Eur. Heart J. 41:111, which is a preferred current guideline) reduced the atherosclerotic burden and local inflammatory activity despite maximum (tolerated) medical treatment and on an unexpectedly large scale. In particular, olamkicept can reduce intima media thickness (IMT), atherosclerotic plaques, and arterial wall inflammation, as measured by cellular infiltration of atherosclerotic plaques.

The invention is therefore suitable for use in the treatment of human patients with ASCVD, preferably high-risk ASCVD, more preferably very-high-risk ASCVD, wherein the human patients preferably are non-responders to treatment with or intolerant to treatment with one or more of statin, ezetimibe, a PCSK9 inhibitor (preferably antibodies such as alirocumab and evolocumab, or short interfering RNA, such as inclisiran), or lipid apheresis therapy.

As used herein, “non-responders” are human patients who show a partial or complete lack of the expected response to an appropriate therapy at an appropriate dose according to current guidelines, whether alone or in combination with other therapies. For example, a biomarker for a non-response to statins, ezetimibe and/or PCSK9 inhibitors is the insufficient reduction or lack of reduction in LDL cholesterol levels in blood and/or plasma and/or serum. The current LDL cholesterol treatment targets for ASCVD are defined, e.g., by the 2019 ESC/EAS Guidelines (Mach et al. 2020, Eur. Heart J. 41:111). The potency of LDL cholesterol-reducing drugs differs not only between drug classes, but may also vary within the same drug class, as observed with the differential efficacy of statins that reduce LDL cholesterol in a range of approximately 30% to 55% at the same maximum dose of 80 mg (Illingworth 2000, Med. Clin. North Am. 84:23). When added to simvastatin therapy, ezetimibe can be expected to further reduce LDL cholesterol by up to approximately 25% (Cannon et al. 2015, N. Engl. J. Med. 372:2387). Anti-PCSK9 antibodies can be expected to reduce LDL cholesterol in addition to statin therapy by approximately 60% (Sabatine et al. 2017, N. Engl. J. Med. 376:1713; Schwartz et al. 2018, N. Engl. J. Med. 379:2097). Therefore, the definition of non-response in a particular patient (group) depends on the type and dose of medication and, if applicable, concomitant medication, but can be determined by a person skilled in the art, such as the attending physician, based on objective guidelines and publicly available literature sources.

Accordingly, a human patient according to the invention can be a patient who, prior to receiving the polypeptide dimer for use in the treatment according to the invention, had received statins, ezetimibe and/or PCSK9 inhibitors. Preferably, a human patient who is a non-responder to treatment with statins, ezetimibe and/or PCSK9 inhibitors, e.g. when using current guidelines, the dosing recommendations of the respective drugs, and/or the outcomes of clinical trials investigating changes of LDL cholesterol levels under treatment with the respective drugs, does not show a reduction of blood levels of LDL cholesterol and/or plasma levels of LDL cholesterol and/or serum levels of LDL cholesterol to the extent that could be expected according to current guidelines, the dosing recommendations of the respective drugs, and/or the outcomes of clinical trials investigating changes of LDL cholesterol levels under treatment with the respective drugs.

As used herein, “intolerance” refers to a partial or complete intolerance of medications, necessitating dose reduction or discontinuation of treatment. Side effects can vary between different drugs of the same class. For example, the most common statin side effects include muscle aches, tenderness or weakness (statin-associated muscle symptoms); headache; dizziness; gastrointestinal problems; fatigue/asthenia; sleep problems; pruritus; elevated liver enzyme levels; or low blood platelet counts. Similar side effects are observed with ezetimibe. Frequently observed side effects during therapy with antibodies directed against PCSK9 (e.g. evolocumab) are flu-like symptoms, vomiting, upper respiratory tract infections as well as back and joint pain. Combinations of several of the above medications may also lead to combinations of side effects and insufficient tolerance and compliance of the patient, resulting in suboptimal maximum tolerated treatment of ASCVD.

The administration of olamkicept according to the invention shows a different, mainly anti-inflammatory mechanism of action and a very favourable side effect profile, which is advantageous, particularly in view of the surprisingly strong therapeutic effect of olamkicept on very-high-risk ASCVD demonstrated in the exemplification.

Human patients with ASCVD to be treated with gp130-Fc fusion peptides such as olamkicept may suffer, e.g., from LDL cholesterol-driven ASCVD, triglyceride-driven ASCVD, lipoprotein a-driven ASCVD, chronic inflammatory disease-driven ASCVD, inflammatory ASCVD, familial hypercholesterolemia, chronic kidney disease, diabetes mellitus, blood pressure greater than 180/110 mm Hg, or human immunodeficiency virus infection.

### EXEMPLIFICATION

### Example 1: Administration of Olamkicept in Treatment of Human Patients Diagnosed with Very-High-Risk ASCVD

As a representative of a polypeptide dimer comprising two gp130-Fc fusion peptides, olamkicept (600 mg intravenously [i.v.] biweekly for 6 and 10 weeks, respectively) was administered to two patients who were suffering from very-high-risk ASCVD despite optimal treatment. The administration of olamkicept was found to reduce IMT, plaque size, and arterial wall inflammation to an unexpected extent in these patients.

Drug Administration:

Olamkicept (produced by Ferring Pharmaceuticals A/S; Copenhagen, Denmark) was administered at a clinical trial dose of 600 mg i.v. within 1 hour, biweekly for 6 weeks (total of 4 infusions) to Patient 1 and for 10 weeks (total of 6 infusions) to Patient 2. Olamkicept's half-life is 4.7 days. Patients were monitored for infusion reactions for 3 hours (first 2 infusions) or 1 hour (subsequent infusions).

Prestudy Evaluation and Phenotypes of the Patients:

Patient characteristics are detailed in Table 1. Patient 1 was a Caucasian male aged 42 years (body mass index [BMI]: 37 kg/m², blood pressure: 140/95 mmHg), with very-high-risk ASCVD (negative for anti-nuclear antibodies [ANA] and anti-neutrophil cytoplasmic antibodies [ANCA]). The patient had a history of recurrent stroke and was under maximum medical treatment consisting of evolocumab, atorvastatin, aspirin, metoprolol, amlodipine, hydrochlorothiazide, doxasozin, and vitamin D. Patient 2 was a Caucasian female aged 64 years (BMI: 37 kg/m², blood pressure 135/90 mmHg), also with very-high-risk ASCVD (ANA/ANCA-negative). She had a history of coronary artery disease and had previously undergone right carotid endarterectomy. The patient's medication consisted of evolocumab, aspirin, metoprolol, amlodipine, hydrochlorothiazide, candesartan, pantoprazole, and vitamin D. Despite maximum tolerated treatment, both patients had a very high risk for future vascular events related to their advanced stage of ASCVD.

Imaging of Atherosclerosis:

For clinical assessment and non-invasive imaging, ultrasound and ¹⁸fluorodeoxyglucose positron emission tomography/computed tomography (¹⁸FDG PET/CT) was used. In Patient 1, screening for ASCVD included an ultrasound examination of the carotid arteries and of the abdominal aorta. The carotid arteries on both sides were scanned with a 7.5 MHz frequency probe in the B-mode, pulsed Doppler mode, and color mode proximal to the carotid bifurcation, in the bifurcation, and in the internal and external carotid arteries. IMT of the arterial wall was evaluated in plaque-free parts, 1 cm proximal to the bulbus of the common carotid artery. The abdominal aorta was scanned with a 5 MHz frequency to detect atherosclerotic plaques. The IMT measured by ultrasound can predict cardiovascular outcomes (Polak et al. 2011, N. Engl. J. Med. 365:213). In Patient 2, screening for inflammatory ASCVD consisted of an ¹⁸FDG PET/CT examination. ¹⁸FDG PET/CT has shown great potential in visualizing, quantifying, and characterizing atherosclerotic inflammation non-invasively, emerging as a suitable surrogate endpoint for clinical testing of novel anti-atherosclerotic therapeutics (Tarkin et al. 2014, Nat. Rev. Cardiol. 11:443). The target-to-background ratio (TBR) was calculated as described previously by van Wijk et al. 2014, J. Am. Coll. Cardiol. 64:1418).

Safety and Metabolic Parameters:

In the two patients with ASCVD, 600 mg olamkicept administered biweekly over 6 weeks (Patient 1) and 10 weeks (Patient 2) was safe. No clinical or laboratory side effects were observed during or after treatment (Table 1). While sIL-6R levels remained unchanged, concentrations of serum IL-6 increased slightly, reflecting olamkicept's additional sgp130 buffering capacity for IL-6/sIL-6R complexes (Table 1). Administration of olamkicept did not change the normal high-sensitivity C-reactive protein (hsCRP) serum levels in Patient 1, but transiently decreased elevated hsCRP by 64-70% 3 days after infusion and by 50% 7 days after infusion in Patient 2 (Table 2). As expected for selective inhibition of IL-6 trans-signaling, serum levels of total cholesterol, high-density lipoprotein (HDL) cholesterol, LDL cholesterol, triglycerides and lipoprotein (a) [(Lp(a)] did not show any clear trends or changes under olamkicept treatment (Table 2). This is in contrast to the common anabolic side effects (increased serum triglyceride and cholesterol levels as well as body weight) observed with anti-IL-6 or anti-IL-6R, which inhibit both classic and trans-signaling (Garbers et al. 2018, Nat. Rev. Drug Discov. 17:395).

Efficacy of Olamkicept Treatment:

In Patient 1 with an LDL cholesterol- and Lp(a)-driven atherosclerosis (Table 2), the IMT in the carotid arteries was slightly increased, and an atherosclerotic plaque was detected in the abdominal aorta (FIG. 1). Four biweekly infusions of olamkicept reduced the IMT from 0.93 to 0.86 mm in the right carotid artery and from 0.98 to 0.89 mm in the left carotid artery (3 months vs. baseline) (FIG. 1A, B). In addition, the atherosclerotic plaque in the abdominal aorta completely resolved under olamkicept treatment (FIG. 1C, D).

Patient 2 presented with an LDL cholesterol-, Lp(a)- and hsCRP-driven atherosclerosis. Therefore, ¹⁸FDG PET/CT images of arterial wall inflammation in the carotid arteries before and after administration of olamkicept (6 biweekly infusions, Table 2) were compared. The density of plaque macrophages has been shown to correlate with the uptake of ¹⁸FDG measured by PET (Tarkin et al. 2014, Nat. Rev. Cardiol. 11:443), and the resulting signal is expressed as mean and maximum target-to-background ratio (TBRₘₑₐₙ and TBRₘₐₓ). The arterial wall inflammation detected by ¹⁸FDG PET/CT at baseline was strongly reduced after 3 months by 6 infusions of olamkicept (FIG. 2).

Taken together, the specific therapeutic inhibition of IL-6 trans-signaling in established ASCVD reduced both the atherosclerotic burden and the local inflammatory activity in the two human patients with very-high-risk ASCVD despite maximum medical treatment and on an unexpectedly large scale.

Patient 1 did not display elevated CRP serum levels. Nevertheless, the anti-cytokine treatment olamkicept surprisingly reduced the IMT and atherosclerotic plaque burden. Accordingly, an elevated CRP level indicating inflammatory activity may not be necessary as a biomarker for patient selection for the use of olamkicept for the treatment of ASCVD.

The specificity and efficacy of olamkicept as a trans-signaling inhibitor was underlined by the absence of changes in lipid levels, especially of Lp(a) (Table 2). As olamkicept does not directly inhibit the induction of acute phase proteins like CRP (Hoge et al. 2013, J. Immunol. 190:703), the decrease of hsCRP in Patient 2 is currently interpreted to reflect the reduction of disease activity in the atherosclerotic lesions.

### FIGURE LEGENDS

FIG. 1: Inhibition of IL-6 trans-signaling reduces intima media thickness and atherosclerotic plaque size in end-stage atherosclerosis. The figure shows representative images of the ultrasound evaluation of Patient 1 at baseline and 12 weeks after the beginning of olamkicept treatment (4 infusions of 600 mg i.v. biweekly; Table 1); (A) Intima media thickness (IMT) before therapy: right carotid artery 0.93 mm, left 0.98 mm (not shown); (B) IMT after therapy: right 0.86 mm, left 0.89 mm (not shown); (C) Abdominal aorta before therapy showing an atherosclerotic plaque; (D) Same site of the abdominal aorta after resolution of the atherosclerotic plaque under olamkicept treatment.

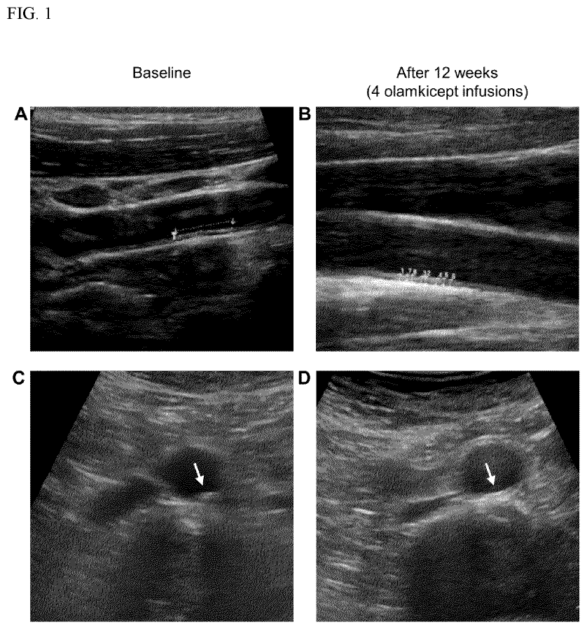

FIG. 2: Inhibition of IL-6 trans-signaling reduces arterial wall inflammation and macrophage infiltration of atherosclerotic plaques in end-stage atherosclerosis. The figure shows arterial wall inflammation in the carotid arteries of Patient 2 (A) at baseline and (B) 11 weeks after the beginning of olamkicept treatment (6 infusions of 600 mg i.v. biweekly; Table 1). In the representative axial computed tomography (CT), ¹⁸fluorodeoxyglucose positron emission tomography (¹⁸FDG PET), and fused images (¹⁸FDG PET/CT), regions of interest are highlighted by bold circles (artery) and narrow circles (vein). Mean and maximum target-to-background ratio (TBRₘₑₐₙ and TBRₘₐₓ) are listed below.

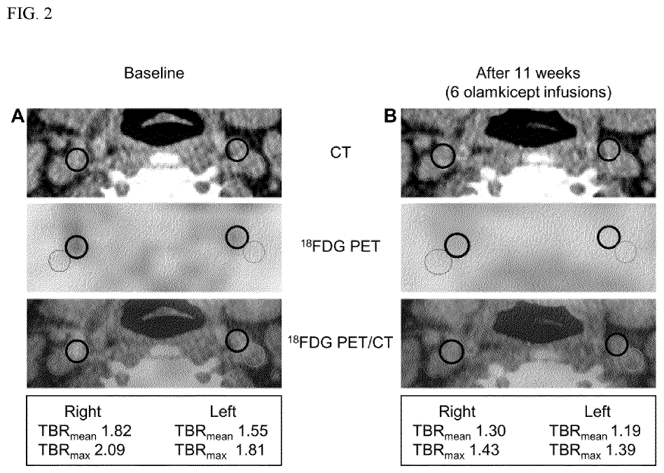

TABLE 1
Patient characteristics

| Days | 0 | 3 | 7 | 14 | 21 | 28 | 42 | 56 | 70 | 73 | 77 | 84 |
|---|---|---|---|---|---|---|---|---|---|---|---|---|
| | Patient 1 | | | | | | | | | | | |
| Leukocytes [×10⁹/L] | 6.2 | 8.16 | 7.16 | 9.03 | — | 6.04 | 6.02 | — | — | — | 6.77 | — |
| Hemoglobin [g/dL] | 15.5 | 15.3 | 15.1 | 14.9 | — | 15.0 | 15.0 | — | — | — | 15.6 | — |
| Platelet [×10⁹/L] | 168 | 171 | 166 | 166 | — | 140 | 151 | — | — | — | 177 | — |
| International Normalized Ratio | 0.95 | 0.98 | 0.99 | 0.99 | — | 1.03 | 0.98 | — | — | — | 0.95 | — |
| D-dimer [mg/L] | 0.24 | <0.22 | <0.22 | 0.32 | — | 0.28 | 0.31 | — | — | — | 0.28 | — |
| Sodium [mmol/L] | 138 | 140 | 139 | 139 | — | 139 | 138 | — | — | — | 139 | — |
| Potassium [mmol/L] | 3.67 | 3.56 | 3.87 | 3.59 | — | 3.44 | 3.52 | — | — | — | 3.94 | — |
| Calcium, albumin- | 2.14 | 2.1 | 2.21 | 2.15 | — | — | — | — | — | — | 2.23 | — |
| corrected [mmol/L] | | | | | | | | | | | | |
| Glomerular filtration | 76 | 75 | 73 | 69 | — | 82 | 73 | — | — | — | 79 | — |
| rate [mL/min/1.73] | | | | | | | | | | | | |
| Apolipoprotein B [mg/dL] | 46 | 43 | 55 | 60 | — | 55 | 62 | — | — | — | 59 | — |
| Bilirubin [μmol/L] | 25.5 | 24.7 | 29.3 | 26.4 | — | 35.5 | 31.1 | — | — | — | 26.4 | — |
| Creatine kinase [U/L] | 197 | 204 | 270 | 251 | — | 294 | 294 | — | — | — | 227 | — |
| Alanine amino-transferase [U/L] | 42.0 | 39.3 | 46.7 | 43.1 | — | 51.1 | 56.1 | — | — | — | 36.2 | — |
| γ-glutamyl transferase [U/L] | 20 | 21 | 23 | 23 | — | 21 | 21 | — | — | — | 22 | — |
| Lipase [U/L] | 32 | 34 | 27 | 26 | — | 26 | 24 | — | — | — | 27 | — |
| Interleukin-6 [pg/mL] | <1.5 | 4.1 | 3.6 | 2.6 | — | 2.8 | 3.7 | — | — | — | <1.5 | — |
| Soluble interleukin-6 receptor | 40.13 | — | — | 37.95 | — | 38.18 | 35.78 | — | — | — | 42.53 | — |
| [ng/mL] | | | | | | | | | | | | |
| Soluble gp130 (including | 485.53 | — | — | 808.95 | — | 990.12 | 1019.64 | — | — | — | 596.91 | — |
| olamkicept) [ng/mL] | | | | | | | | | | | | |
| | Patient 2 | | | | | | | | | | | |
| Leukocytes [×10⁹/L] | 10.7 | 10.4 | — | 10.2 | 10.8 | 11.0 | 10.7 | 11.9 | 10.3 | 8.54 | 11.0 | — |
| Hemoglobin [g/dL] | 15.6 | 16.7 | — | 15.7 | 16.1 | 15.7 | 15.3 | 15.5 | 15.9 | 15.6 | 16.1 | — |
| Platelet [×10⁹/L] | 200 | 203 | — | 173 | 179 | 193 | 175 | 178 | 195 | 84 | 191 | — |
| International Normalized Ratio | 1.01 | — | — | 1.02 | 0.99 | 096 | 1.05 | 1.01 | 1.06 | — | — | — |
| D-dimer \|mg/L] | 0.34 | — | — | 0.31 | 0.34 | 0.47 | 0.32 | 0.26 | 0.32 | — | — | — |
| Sodium [mmol/L] | 141 | 140 | — | 143 | 143 | 139 | 140 | 144 | 140 | — | — | — |
| Potassium [mmol/L] | 3.84 | 3.75 | — | 3.91 | 3.99 | 4.05 | 3.94 | 4.03 | 3.92 | — | — | — |
| Calcium, albumin- | 2.38 | 2.44 | — | 2.44 | — | — | — | 2.54 | 2.49 | — | 2.4 | — |
| corrected [mmol/L] | | | | | | | | | | | | |
| Glomerular filtration | 60 | 68 | — | 60 | 67 | 73 | 63 | 60 | 63 | — | — | — |
| rate [mL/min/1.73] | | | | | | | | | | | | |
| Apolipoprotein B [mg/dL] | 51 | — | — | 56.0 | 61.0 | 53.0 | 51.0 | 51.0 | 53.0 | — | 66.0 | — |
| Bilirubin [μmol/L] | 7.4 | 8.3 | — | 9.1 | 9.0 | 7.4 | 8.5 | 5.1 | 8.8 | — | — | — |
| Creatine kinase [U/L] | 42 | 37 | — | 28 | 37 | 30 | 27 | 54 | 35 | — | — | — |
| Alanine amino-transferase [U/L] | 13.8 | 15.5 | — | 15.0 | 16.8 | 13.0 | 19.0 | 15.9 | 13.8 | — | — | — |
| γ-glutamyl transferase [U/L] | 40 | 41 | — | 32 | 39 | 39 | 37 | 38 | 40 | — | — | — |
| Lipase [U/L] | 33 | 41 | — | 33 | 34 | 36 | 32 | 29 | 33 | — | — | — |
| Interleukin-6 [pg/mL] | 4.8 | 32.0 | — | 9.8 | 24.3 | 12.0 | 11.7 | 15.5 | 11.7 | 40.6 | 26.8 | — |
| Soluble interleukin-6 receptor | 60.84 | — | — | 61.18 | — | 66.67 | 70.45 | 53.86 | 60.84 | 64.15 | 54.54 | — |
| [ng/mL] | | | | | | | | | | | | |
| Soluble gp130 (including | 319.12 | — | — | 983.41 | — | 1094.79 | 1281.33 | 1061.24 | 1014.27 | 2620.65 | 1865.10 | — |
| olamkicept) [ng/mL] | | | | | | | | | | | | |

TABLE 2
Treatment schedule, diagnostics, and metabolic parameters

| Days | 0 | 3 | 7 | 14 | 21 | 28 | 42 | 56 | 70 | 73 | 77 | 84 |
|---|---|---|---|---|---|---|---|---|---|---|---|---|
| | Patient 1 | | | | | | | | | | | |
| Olamkicept | x | — | — | x | — | x | x | — | — | — | — | — |
| Ultrasound | x | — | — | — | — | — | — | — | — | — | — | x |
| Cholesterol [mmol/L] | 2.8 | 2.9 | 3.2 | 3.6 | — | 3.1 | 3.3 | — | — | — | 3.2 | — |
| HDL (high-density lipoprotein) | 1.59 | 1.48 | 1.65 | 1.65 | — | 1.35 | 1.6 | — | — | — | 1.56 | — |
| cholesterol [mmol/L] | | | | | | | | | | | | |
| LDL (low-density lipoprotein) | 1.32 | 1.21 | 1.57 | 1.89 | — | 1.55 | 1.79 | — | — | — | 1.65 | — |
| cholesterol [mmol/L] | | | | | | | | | | | | |
| Triglycerides [mmol/L] | 0.8 | 1.8 | 0.8 | 0.9 | — | 1.0 | 0.8 | — | — | — | 1.1 | — |
| Lipoprotein (a) [nmol/L] | 296.6 | 267.1 | 276.7 | 291.3 | — | 336.7 | 283.7 | — | — | — | 303.1 | — |
| high-sensitivity C-reactive protein | 0.37 | <0.3 | 0.6 | <0.3 | — | 0.37 | 0.68 | — | — | — | 0.65 | — |
| [mg/dL] | | | | | | | | | | | | |
| HbA1c (glycated haemoglobin) [%] | 5.0 | — | — | — | — | — | — | — | — | — | 5.2 | — |
| | Patient 2 | | | | | | | | | | | |
| Olamkicept | x | — | — | x | — | x | x | x | x | — | — | — |
| PET/CT | x | — | — | — | — | — | — | — | — | — | x | — |
| Cholesterol [mmol/L] | 2.7 | 3.1 | — | 2.9 | 3.1 | 2.9 | 2.6 | 2.6 | 2.7 | — | 3.0 | — |
| HDL (high-density lipoprotein) | 1.0 | 1.15 | — | 1.08 | 1.05 | 1.03 | 0.97 | 1.04 | 0.97 | — | 1.19 | — |
| cholesterol [mmol/L] | | | | | | | | | | | | |
| LDL (low-density lipoprotein) | 1.3 | 1.65 | — | 1.4 | 1.62 | 1.36 | 1.21 | 1.27 | 1.34 | — | 1.48 | — |
| cholesterol [mmol/L] | | | | | | | | | | | | |
| Triglycerides [mmol/L] | 2.2 | 2.2 | — | 2.0 | 2.4 | 2.0 | 2.2 | 2.1 | 2.2 | — | 1.7 | — |
| Lipoprotein (a) [nmol/L] | 238.7 | 250.2 | — | 227.2 | 236.7 | 241.1 | 234.2 | 225.7 | 243.0 | — | 231.7 | — |
| high-sensitivity C-reactive protein | 9.18 | 3.33 | — | 12.7 | 7.06 | 9.99 | 11.2 | 18.9 | 14.4 | 4.1 | 7.14 | — |
| [mg/dL] | | | | | | | | | | | | |
| HbA1c (glycated haemoglobin) [%] | 6.7 | — | — | — | — | — | — | — | — | — | 6.5 | — |

SEQUENCE LISTING

```
<210> 1
<211> 822
<212> PRT
<213> Artificial Sequence
<220>
<223> polypeptide dimer comprising two gp130-Fc fusion peptides
<220>
<221> CHAIN
<222> 585 . . . 595
<223> part of gp130 D6 domain
<220>
<221> CHAIN
<222> 690 . . . 612
<223> part of Fc domain hinge region
<400> 1
Glu Leu Leu Asp Pro Cys Gly Tyr Ile Ser Pro Glu Ser Pro Val Val
1 5 10 15
Gln Leu His Ser Asn Phe Thr Ala Val Cys Val Leu Lys Glu Lys Cys
                        20 25 30
Met Asp Tyr Phe His Val Asn Ala Asn Tyr Ile Val Trp Lys Thr Asn
                35 40 45
His Phe Thr Ile Pro Lys Glu Gln Tyr Thr Ile Ile Asn Arg Thr Ala
        50 55 60
Ser Ser Val Thr Phe Thr Asp Ile Ala Ser Leu Asn Ile Gln Leu Thr
65 70 75 80
Cys Asn Ile Leu Thr Phe Gly Gln Leu Glu Gln Asn Val Tyr Gly Ile
                                85 90 95
Thr Ile Ile Ser Gly Leu Pro Pro Glu Lys Pro Lys Asn Leu Ser Cys
                        100 105 110
Ile Val Asn Glu Gly Lys Lys Met Arg Cys Glu Trp Asp Gly Gly Arg
                115 120 125
Glu Thr His Leu Glu Thr Asn Phe Thr Leu Lys Ser Glu Trp Ala Thr
        130 135 140
His Lys Phe Ala Asp Cys Lys Ala Lys Arg Asp Thr Pro Thr Ser Cys
145 150 155 160
Thr Val Asp Tyr Ser Thr Val Tyr Phe Val Asn Ile Glu Val Trp Val
                                165 170 175
Glu Ala Glu Asn Ala Leu Gly Lys Val Thr Ser Asp His Ile Asn Phe
                        180 185 190
Asp Pro Val Tyr Lys Val Lys Pro Asn Pro Pro His Asn Leu Ser Val
                195 200 205
Ile Asn Ser Glu Glu Leu Ser Ser Ile Leu Lys Leu Thr Trp Thr Asn
        210 215 220
Pro Ser Ile Lys Ser Val Ile Ile Leu Lys Tyr Asn Ile Gln Tyr Arg
225 230 235 240
Thr Lys Asp Ala Ser Thr Trp Ser Gln Ile Pro Pro Glu Asp Thr Ala
                                245 250 255
Ser Thr Arg Ser Ser Phe Thr Val Gln Asp Leu Lys Pro Phe Thr Glu
                        260 265 270
Tyr Val Phe Arg Ile Arg Cys Met Lys Glu Asp Gly Lys Gly Tyr Trp
                275 280 285
Ser Asp Trp Ser Glu Glu Ala Ser Gly Ile Thr Tyr Glu Asp Arg Pro
        290 295 300
Ser Lys Ala Pro Ser Phe Trp Tyr Lys Ile Asp Pro Ser His Thr Gln
305 310 315 320
Gly Tyr Arg Thr Val Gln Leu Val Trp Lys Thr Leu Pro Pro Phe Glu
                                325 330 335
Ala Asn Gly Lys Ile Leu Asp Tyr Glu Val Thr Leu Thr Arg Trp Lys
                        340 345 350
Ser His Leu Gln Asn Tyr Thr Val Asn Ala Thr Lys Leu Thr Val Asn
                355 360 365
Leu Thr Asn Asp Arg Tyr Leu Ala Thr Leu Thr Val Arg Asn Leu Val
        370 375 380
Gly Lys Ser Asp Ala Ala Val Leu Thr Ile Pro Ala Cys Asp Phe Gln
385 390 395 400
Ala Thr His Pro Val Met Asp Leu Lys Ala Phe Pro Lys Asp Asn Met
                                405 410 415
Leu Trp Val Glu Trp Thr Thr Pro Arg Glu Ser Val Lys Lys Tyr Ile
                        420 425 430
Leu Glu Trp Cys Val Leu Ser Asp Lys Ala Pro Cys Ile Thr Asp Trp
                435 440 445
Gln Gln Glu Asp Gly Thr Val His Arg Thr Tyr Leu Arg Gly Asn Leu
        450 455 460
Ala Glu Ser Lys Cys Tyr Leu Ile Thr Val Thr Pro Val Tyr Ala Asp
465 470 475 480
Gly Pro Gly Ser Pro Glu Ser Ile Lys Ala Tyr Leu Lys Gln Ala Pro
                                485 490 495
Pro Ser Lys Gly Pro Thr Val Arg Thr Lys Lys Val Gly Lys Asn Glu
                        500 505 510
Ala Val Leu Glu Trp Asp Gln Leu Pro Val Asp Val Gln Asn Gly Phe
                515 520 525
Ile Arg Asn Tyr Thr Ile Phe Tyr Arg Thr Ile Ile Gly Asn Glu Thr
        530 535 540
Ala Val Asn Val Asp Ser Ser His Thr Glu Tyr Thr Leu Ser Ser Leu
545 550 555 560
Thr Ser Asp Thr Leu Tyr Met Val Arg Met Ala Ala Tyr Thr Asp Glu
                                565 570 575
Gly Gly Lys Asp Gly Pro Glu Phe Thr Phe Thr Thr Pro Lys Phe Ala
                        580 585 590
Gln Gly Glu Asp Lys Thr His Thr Cys Pro Pro Cys Pro Ala Pro Glu
                595 600 605
Ala Glu Gly Ala Pro Ser Val Phe Leu Phe Pro Pro Lys Pro Lys Asp
        610 615 620
Thr Leu Met Ile Ser Arg Thr Pro Glu Val Thr Cys Val Val Val Asp
625 630 635 640
Val Ser His Glu Asp Pro Glu Val Lys Phe Asn Trp Tyr Val Asp Gly
                                645 650 655
Val Glu Val His Asn Ala Lys Thr Lys Pro Arg Glu Glu Gln Tyr Asn
                        660 665 670
Ser Thr Tyr Arg Val Val Ser Val Leu Thr Val Leu His Gln Asp Trp
                675 680 685
Leu Asn Gly Lys Glu Tyr Lys Cys Lys Val Ser Asn Lys Ala Leu Pro
        690 695 700
Ala Pro Ile Glu Lys Thr Ile Ser Lys Ala Lys Gly Gln Pro Arg Glu
705 710 715 720
Pro Gln Val Tyr Thr Leu Pro Pro Ser Arg Glu Glu Met Thr Lys Asn
                                725 730 735
Gln Val Ser Leu Thr Cys Leu Val Lys Gly Phe Tyr Pro Ser Asp Ile
                        740 745 750
Ala Val Glu Trp Glu Ser Asn Gly Gln Pro Glu Asn Asn Tyr Lys Thr
                755 760 765
Thr Pro Pro Val Leu Asp Ser Asp Gly Ser Phe Phe Leu Tyr Ser Lys
        770 775 780
Leu Thr Val Asp Lys Ser Arg Trp Gln Gln Gly Asn Val Phe Ser Cys
785 790 795 800
Ser Val Met His Glu Ala Leu His Asn His Tyr Thr Gln Lys Ser Leu
                                805 810 815
Ser Leu Ser Pro Gly Lys
                        820
<210> 2
<211> 11
<212> PRT
<213> Artificial Sequence
<220>
<223> part of gp130 D6 domain, amino acids No 585 . . . 595 of SEQ ID
NO: 1
<400> 2
Thr Phe Thr Thr Pro Lys Phe Ala Gln Gly Glu
1 5 10
<210> 3
<211> 4
<212> PRT
<213> Artificial Sequence
<220>
<223> part of Fc domain hinge region, amino acids No 609 . . . 612 of
SEQ ID NO: 1
<400> 3
Ala Glu Gly Ala
1
```

1. A polypeptide dimer comprising two gp130-Fc monomers, each monomer having at least 90% sequence identity to SEQ ID NO: 1, for use in the treatment of human patients with atherosclerotic cardiovascular disease (ASCVD).

2. The polypeptide dimer according to claim 1, for use in the manufacture of a medicament for treatment of human patients with ASCVD.

3. The polypeptide dimer for use according to any one of the preceding claims, wherein the ASCVD is very-high-risk ASCVD.

4. The polypeptide dimer for use according to any one of the preceding claims, wherein the monomers comprise the gp130 D6 domain comprising the amino acids at positions 585-595 of SEQ ID NO:1, an Fc domain hinge region comprising the amino acids at positions 609-612 of SEQ ID NO:1, and the monomers do not comprise a linker between the gp130 part and the Fc part.

5. The polypeptide dimer according to any one of the preceding claims, for use in the treatment of human patients with ASCVD, characterized in that the human patients are non-responders to treatment with or intolerant to treatment with one or more of a statin, ezetimibe, and an inhibitor of proprotein convertase subtilisin/kexin type 9 (PCSK9 inhibitor).

6. The polypeptide dimer for use in treatment according to any one of the preceding claims, characterized in that the human patient does not respond to or is intolerant to a combination of a statin and ezetimibe.

7. The polypeptide dimer for use in treatment according to any one of the preceding claims, characterized in that the human patient does not respond to or is intolerant to a combination of a statin and a PCSK9 inhibitor.

8. The polypeptide dimer for use in treatment according to any one of the preceding claims, characterized in that the human patient does not respond to or is intolerant to a combination of ezetimibe and a PCSK9 inhibitor.

9. The polypeptide dimer for use in treatment according to any one of the preceding claims, characterized in that the human patient does not respond to or is intolerant to a combination of a statin, ezetimibe, and a PCSK9 inhibitor.

10. The polypeptide dimer for use in treatment according to any one of the preceding claims, characterized in that the human patient is classified as a non-responder to one or more of a statin, ezetimibe, and a PCSK9 inhibitor based upon detection of a biomarker for non-response.

11. The polypeptide dimer for use in treatment according to any one of the preceding claims, characterized in that the biomarker for non-response to treatment with one or more of a statin, ezetimibe, and a PCSK9 inhibitor, is the insufficient reduction of blood levels of LDL cholesterol and/or plasma levels of LDL cholesterol and/or serum levels of LDL cholesterol compared to objective expectations based on the treatment targets in current guidelines, the dosing recommendations of the respective drugs, and/or the outcomes of clinical trials investigating changes of LDL cholesterol levels under treatment with the respective drugs.

12. The polypeptide dimer for use in treatment according to any one of the preceding claims, characterized in that the human patient does not respond to or is intolerant to lipid apheresis therapy.

13. The polypeptide dimer for use in treatment according to any one of the preceding claims, characterized in that the use reduces one or more of atherosclerotic plaque size, intima media thickness, and arterial wall inflammation.

14. The polypeptide dimer for use in treatment according to any one of the preceding claims, characterized in that the ASCVD is low density lipoprotein-driven ASCVD, triglyceride-driven ASCVD, lipoprotein a-driven ASCVD, chronic inflammatory disease-driven ASCVD, or inflammatory ASCVD.

15. The polypeptide dimer for use in treatment according to any one of the preceding claims, characterized in that the human patient has one or more of familial hypercholesterolemia, chronic kidney disease, diabetes mellitus, blood pressure greater than 180/110 mm Hg, and human immunodeficiency virus infection.

16. The polypeptide dimer for use in treatment according to any one of the preceding claims, characterized in that the use comprises a dose for administration of 60 mg to 1 g of the polypeptide dimer, preferably 150 to 600 mg.

17. The polypeptide dimer for use in treatment according to any one of the preceding claims, characterized in that the use is for administration once per every 1 to 4 weeks, preferably every 1 to 2 weeks.

18. A method for treating atherosclerotic cardiovascular disease (ASCVD) in a human patient, said method comprising administering to a patient in need thereof a therapeutically effective amount of a polypeptide dimer comprising two gp130-Fc monomers, each monomer having at least 90% sequence identity to SEQ ID NO: 1.


SEQUENCE LISTING (3 sequences)

SEQ ID NO: 1 - protein, 822 aa, Artificial Sequence
  polypeptide dimer comprising two gp130-Fc fusion peptides
  CHAIN (585)..(595): part of gp130 D6 domain
  CHAIN (609)..(612): part of Fc domain hinge region
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 11 aa, Artificial Sequence
  part of gp130 D6 domain, amino acids No 585..595 of SEQ ID NO 1
TFTTPKFAQGE

SEQ ID NO: 3 - protein, 4 aa, Artificial Sequence
  part of Fc domain hinge region, amino acids No 609..612 of SEQ ID NO 1
AEGA